(19) United States
(12) Patent Application Publication
(10) Pub. No.: US 2007/0255088 A1
(43) Pub. Date: Nov. 1, 2007

(54) Implantable, magnetic actuator

(76) Inventors: Andrew D. Jacobson, San Antonio, TX (US); Jeff Sommers, San Antonio, TX (US); Thomas E. Nolan, Harleysville, PA (US)

Correspondence Address: Ms. Soula Skokos, Suite 220, 1100 Jorie Boulevard, Oak Brook, IL, 60523, US

(21) Appl. No.: 11/784,964

(22) Filed: Apr. 10, 2007

Related U.S. Application Data
(60) Provisional application No. 60/791,137, filed on Apr. 11, 2006.

(51) Int. Cl. A61N 2/10

(52) U.S. Cl. 600/12

(57) ABSTRACT

An implantable, magnetic actuator comprising a mechanical switch completely implanted within the human's or animal's body having at least one magnet; an external user component having at least one magnet which is used externally of the animal's or human's body to couple to the magnet of the mechanical switch; a connector that is connected at one end to the mechanical switch and at the opposite end may be connected to activation means that may be used to remotely actuate a mechanism, component or device that has been implanted completely within an animal's or human's body and is located distally from the implantable, magnetic actuator. The mechanical switch, connector and activation means are completely implanted within the human's or animal's body to overcome the risks and obstacles posed by exposed actuators.

### CROSS REFERENCE TO RELATED APPLICATION

The present application is a regular utility application claiming the benefits of the filing date of provisional application, Application No. 60,791,137, filed on Apr. 11, 2006 by the present applicants.

### BACKGROUND OF THE INVENTION

The present invention relates generally to an implantable medical device and more particularly to an implantable actuator having at least one magnet to remotely actuate a medical device that has been implanted completely within an animal's or human's body.

In the field of both veterinary and human medicine, actuators are used to remotely actuate surrounding bodily tissue, to intervene in or monitor the body's activities, or to actuate an implanted medical device which will in turn actuate surrounding bodily tissue or intervene in or monitor the body's activities. Such types of actuators include non-implanted and partially implanted devices such as laparoscopic devices and wire-based interventional catheters. Non-implanted and partially implanted devices, however, do not offer the same benefits as actuators that are implanted within the body including most notably a decreased risk of infection, decreased risk of device dislodgement and damage, reduced maintenance, and improved cosmesis. While fully implanted actuators currently exist, some require transcutaneous needle access to activate or require palpating the animal's or human's skin in order to actuate a non-magnetic switch. Examples of implantable actuators requiring transcutaneous needle access for activation include an implanted injection port utilizing hydraulic or pneumatic power to inflate a polymeric gastric band and an implanted injection port utilizing hydraulic or pneumatic power to inflate a polymeric vascular occluder. Such implantable actuators consequently can cause pain to the human or animal patient or subject.

An actuator that is implanted which does not require transcutaneous needle access or palpation or manipulation of the skin to actuate can overcome these problems and increase comfort to the animal or human and decrease the risk of infection. The present invention of an implanted actuator utilizing magnet force through the skin for actuation is less traumatic and painful than an implanted actuator requiring transcutaneous needle access or manipulation by the human operator to actuate.

The present invention overcomes the problems faced by current actuators by using magnetic forces through the animal's or human's skin to actuate a medical device located distally from the implantable actuator. The present invention requires little or no contact with the human or animal, thus increasing the comfort, or reducing stress, of the human or animal during the actuation process. The implantable, magnetic actuator also gives quick discernable confirmation of its position. Another advantage of the implantable, magnetic actuator is that the body tends to defend against foreign bodies thereby resulting in swelling and fibrosing. The swelling and fibrosing of an implantable actuator that requires palpating, therefore, can potentially impede performance of the actuator and increase pain and stress associated with actuation. The implantable, magnetic actuator, however, may be housed within a casing which reduces the possibility of any complications arising from fibrosis and swelling.

### SUMMARY OF THE INVENTION

The present invention is directed to an implantable, magnetic actuator comprising a mechanical switch completely implanted within the human's or animal's body having at least one magnet; an external user component having at least one magnet which is used externally of the animal's or human's body to couple to the magnet of the mechanical switch; a connector that is connected at one end to the mechanical switch and at the opposite end may be connected to activation means that may be used to remotely actuate a mechanism, component or device that has been implanted completely within an animal's or human's body and is located distally from the implantable, magnetic actuator. In the present invention, the mechanical switch, connector and activation means are completely implanted within the human's or animal's body to overcome the risks and obstacles posed by externalized actuators.

The activation means attach to the mechanical switch via a connector that is attached at one end to the mechanical switch and at the opposite end to the activation means. The activation means of the implantable, magnetic actuator may be but are not limited to a clamp, valve, cutting tool, conductive element, switch, piston, suture, manipulator, balloon, fabric, membrane, hook, stirrup, wire, noose, or any other element, component, or device which is activated to actuate surrounding bodily tissue, to intervene in or monitor the body's activities, or to actuate an implanted medical device which will in turn actuate surrounding bodily tissue or intervene in or monitor the body's activities.

To activate the activation means, the user places the external user component above the surface of skin directly above the mechanical switch of the implantable, magnetic actuator which has been implanted within the human's or animal's body. The external user component therefore does not come into physical contact with the mechanical switch but rather uses magnetic forces to manipulate the mechanical switch. Once the magnet of the external user component and mechanical switch magnet line up, the magnet within the external user component couples with the mechanical switch magnet. By moving the external user component along a certain plane or prescribed path of movement, the mechanical switch magnet having coupled to the magnet of the external user component forces the mechanical switch to move. In the present invention, the mechanical switch may move into various positions, including “deactivated” and “activated” positions and multiple intermediate positions that vary between the “deactivated” position and “activated” position. In the “activated” position, the movement of the mechanical switch and thereby the movement of the connector cause the activation means to actuate surrounding bodily tissue, to intervene in or monitor the body's activities, or to actuate an implanted medical device which will in turn actuate surrounding bodily tissue or intervene in or monitor the body's activities. To place the mechanical switch into the “deactivated” position, the user moves the external user component in the direction opposite of the movement prescribed to place the mechanical switch in the “activated” position. The movement of the mechanical switch in the opposite direction causes the connector to move in the opposite direction thereby causing the activation means to be in their opposite position and now in a “deactivated” position.

One distinct advantage of the present invention is that the user is able to change the position of the mechanical switch by moving the external user component between “deactivated” and “activated” positions without direct contact to the body. While minimum contact between the external user component and the skin overlying the mechanical switch may occur, such contact is minimal wherein the external user component is simply gliding on the surface of the skin. Once the mechanical switch has been placed into a position, the mechanical switch may lock in place, and the external user component may be removed.

In the present invention, the connector may be but is not limited to polymeric or metallic linear wire, polymeric or metallic curvilinear wire, pneumatic tubing, hydraulic tubing, rod, cylinder, wire bundle, string, cord, or spring. The connector may be of a material that is biocompatible and acceptable for subcutaneous and implanted medical devices.

In a preferred embodiment, the external user component is a wand having rod shape and having a magnet at one end.

In a preferred embodiment, the implantable, magnetic actuator may be enclosed in a housing or flexible shell in order to prevent any complications resulting from fibrosis and swelling and to prevent body fluids from interfering with the function of the mechanical switch. The housing or flexible shell may be of a polymer or metal that is biocompatible and acceptable for subcutaneous medical devices.

In another embodiment, the implantable, magnetic actuator may have a sheath that is concentric to the connector and provides a protective barrier and passageway for the connector to pass. The sheath also adds to the functioning of the connector by reinforcing the connector as the mechanical switch is “activated” and the connector becomes fully extended. The sheath may be of a biocompatible polymer or metal to provide the connector with protection and a passageway.

The mechanical switch may be of a polymer or metal that is biocompatible and acceptable for subcutaneous medical devices.

### BRIEF DESCRIPTION OF THE DRAWINGS

FIG. 1a is a top cut away view of the implantable, magnetic actuator designed in accordance with an embodiment of the present invention, the figure illustrating the mechanical switch in an “activated” position;

FIG. 1b is top cut away view of the implantable, magnetic actuator designed in accordance with an embodiment of the present invention, the figure illustrating the mechanical switch in a “deactivated” position;

FIG. 2a is perspective view of the implantable, magnetic actuator designed in accordance with another embodiment of the present invention, the figure illustrating the mechanical switch in an “activated” position;

FIG. 2b is perspective view of the implantable, magnetic actuator designed in accordance with another embodiment of the present invention, the figure illustrating the mechanical switch in a “deactivated” position;

FIG. 3a is top cut away view of the implantable, magnetic actuator designed in accordance with another embodiment of the present invention, the figure illustrating the mechanical switch in an “activated” position; and

FIG. 3b is top cut away view of the implantable, magnetic actuator designed in accordance with another embodiment of the present invention, the figure illustrating the mechanical switch in a “deactivated” position

FIG. 4a is a schematic of the implantable, magnetic actuator in use with a medical device intended for bile collection in a research animal. The mechanical switch is in a “deactivated” position wherein bile is allowed to flow in the normal direction from the gallbladder to the duodenum.

FIG. 4b is a schematic of the implantable, magnetic actuator in use with a medical device intended for bile collection in a research animal. The mechanical switch is in an “activated” position which allows a spring to relax and direct a plug to occlude the artificial bile tube thereby diverting the flow of bile away from the duodenum towards a subcutaneous access port where the bile is sampled.

### DESCRIPTION OF THE EMBODIMENTS

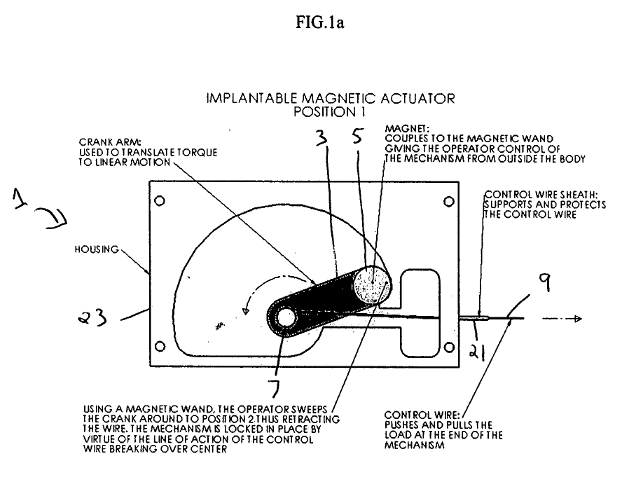

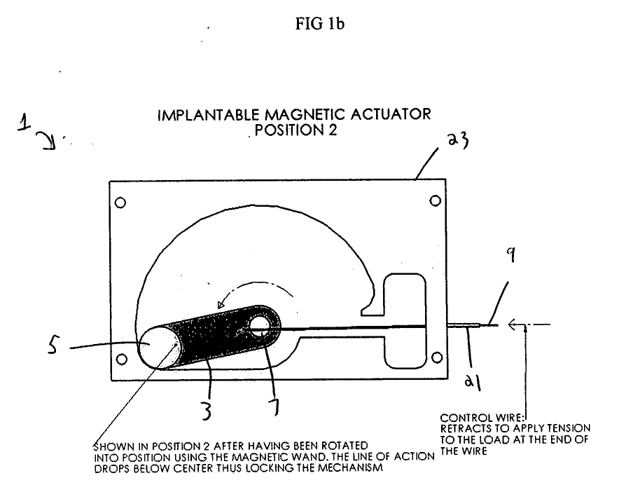

FIGS. 1a and 1b illustrate one embodiment of the invention, wherein the implantable, magnetic actuator, designated generally as 1 in the Figures, is shown in the “activated” position.

In this embodiment, the mechanical switch 3 is a crank arm and the magnet 5 is attached at the end of the mechanical switch 3 opposite the pivot point 7. The connector 9, shown as a control wire, is connected at one end to the mechanical switch 3 near the pivot point 7 of the mechanical switch 3. In the “activated” position, as shown in FIG. 1a, the mechanical switch 3 is most proximal to the connector 9 and the connector 9 is fully extended. In its fully extended position, the connector 9 causes the activation means (not shown) to extend to actuate surrounding bodily tissue, to intervene in or monitor the body's activities, or to actuate an implanted medical device which will in turn actuate surrounding bodily tissue or intervene in or monitor the body's activities. For example, in the “activated” position, the activation means, such as a clamp, may be extended away from the mechanical switch 3 to occlude a desired catheter or biological passageway.

In this embodiment, as the user places the external user component 11 above the surface of the skin directly above magnet 5 of the mechanical switch 3, the magnet 5 and the magnet 13 of the external user component 11 are coupled and the user may reposition the external user component 11 to move the mechanical switch 3 in the direction opposite the connector 9 to cause the mechanical switch 3 to move into the “deactivated” position as shown in FIG. 1b. FIG. 1b illustrates the mechanical switch 3 in the “deactivated” position wherein the mechanical switch 3 is most distal to the connector 9 and the connector 9 is retracted.

The mechanical switch 3 may be reversible, irreversible or adjustable, and may have multiple positions including but not limited to “deactivated” and “activated” positions, as is the case with a two-position switch, and multiple intermediate positions that vary between the “deactivated” position and “activated” position. In the “deactivated” position, as shown in FIG. 1b, the mechanical switch 3 of the implantable, magnetic actuator 1 may be in the position whereby the connector 9 is withdrawn toward the mechanical switch 3 resulting in the activation means being in the “deactivated” position, located proximally to the mechanical switch 3. To cause the mechanical switch 3 to be in the “activated” position, the user places the external user component 11 over the surface of the animal's or human's skin above the magnet 5 in order to couple the magnet 5 and the magnet 13 in the external user component 11. Once the magnet 5 and the magnet 13 in the external user component 11 are coupled, the user then repositions the external user component 11 in the direction of the connector 9 such that the mechanical switch 3 moves towards the connector 9 thereby causing the connector 9 to be fully extended causing the activation means to be in the “activated” position, located distally from the mechanical switch 3.

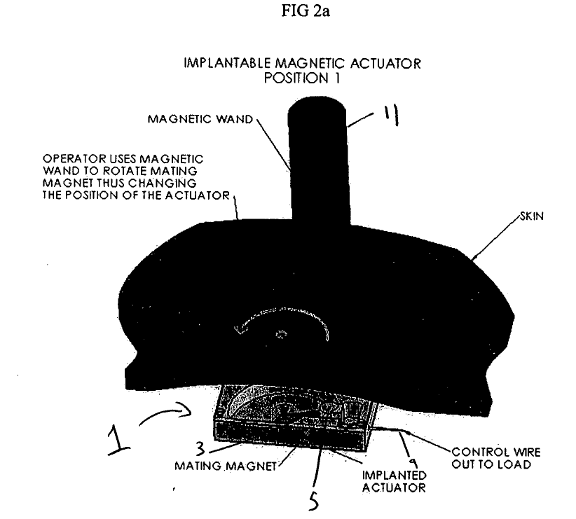

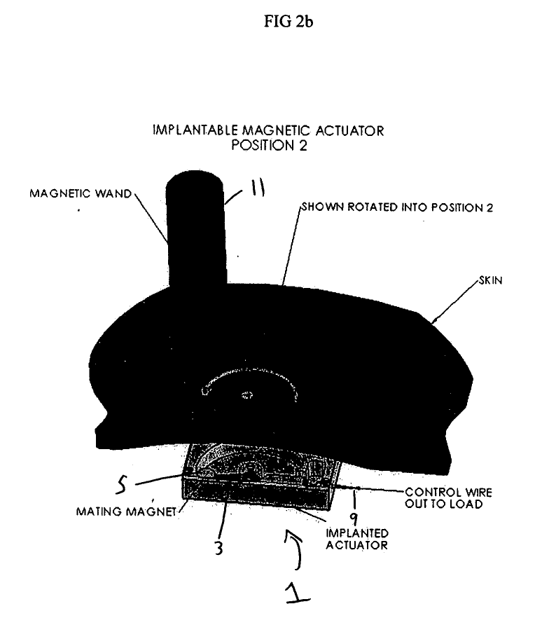

In another embodiment, the mechanical switch 3 of the implantable, magnetic actuator 1 may be inverted wherein the “deactivated” and “activated” positions are reversed as shown in FIGS. 2a and 2b. In an implantable, magnetic actuator where the mechanical switch 3 is inverted and the “deactivated” and “activated” positions are reversed, the withdrawn position of the connector 9 may correspond to the “activated” position wherein the activation means are “activated,” while the fully extended position of the connector 9 may correspond to the “deactivated” position wherein the activation means are “deactivated.”

As shown in FIG. 2a, the mechanical switch 3 is in the “activated” position where the mechanical switch 3 is most proximal to the connector 9 and the connector 9 is fully extended. As the user places the external user component 11 over the surface of the animal's or human's skin above the magnet 5, the magnet 5 and the magnet 13 in the external user component 11 are coupled and the user may reposition the external user component to rotate the magnet 5 thus moving the mechanical switch 3 in the direction opposite the connector 9 in order to cause the mechanical switch 3 to be in the “deactivated” position.

FIG. 2b illustrates the mechanical switch 3 in the “deactivated” position wherein the user has repositioned the external user component 11 to rotate the magnet 5 and cause the mechanical switch 3 to be most distal to the connector 9 and retract the connector 9.

As shown in FIGS. 2a and 2b, the use of the external user component 11 to activate the mechanical switch is non-invasive since the contact with the patient is minimal. The magnetic forces of the magnet 5 and magnet 13 of external user component 11 transpire through the skin thereby causing the patient little discomfort and greatly reducing the rate of infection when compared to exposed actuators.

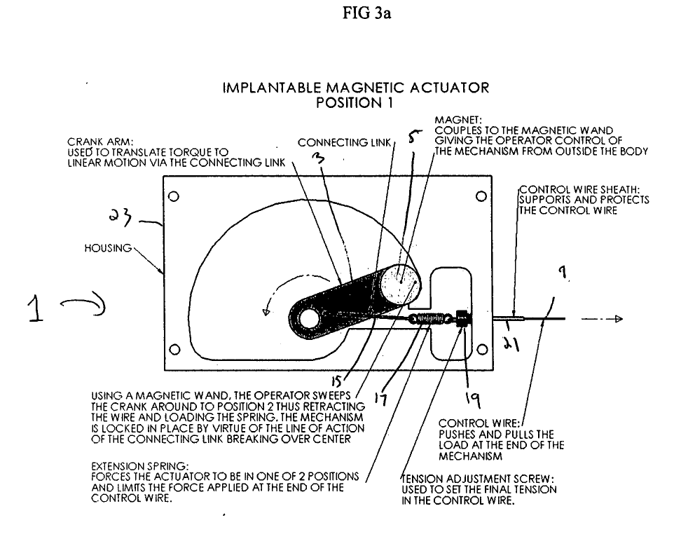

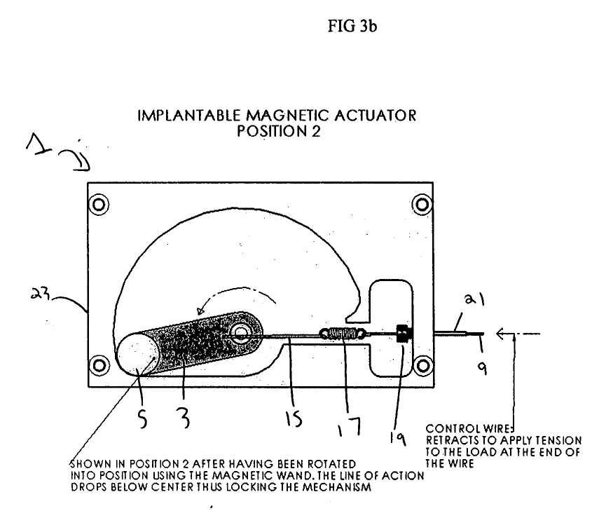

FIG. 3a illustrates another embodiment of the present invention in an “activated” position wherein a connecting link 15 is connected to the mechanical switch 3 at one end and to a spring 17 and adjustment screw 19 at the opposite end. The connector 9 is then connected to the adjustment screw 19 at one end. The connecting link 15 couples the spring 17 and reduces bending of the connector 9, which is shown as a control wire. The spring 17 limits the force imparted to the medical device to be actuated by the implantable, magnetic actuator 1, as well as provide a locking mechanism to the implantable, magnetic actuator 1. Once the mechanical switch 3 is moved into position, the spring 17 locks or secures the mechanical switch 3 to prevent inadvertent changing of positions between “activated” and “deactivated” positions.

FIG. 3b illustrates the mechanical switch 3 in the “deactivated” position wherein the user has repositioned the external user component 11 to rotate the magnet 5 and cause the mechanical switch 3 to be most distal to the connector 9 and retract the connector 9. The adjustment screw 19 takes up the slack in the sheath 21 and controls the final tension in the connector 9 in its retracted position.

In a preferred embodiment, the implantable, magnetic actuator may be enclosed in a housing or flexible shell, as designated in the Figures as numeral 23, in order to prevent any complications resulting from fibrosis.

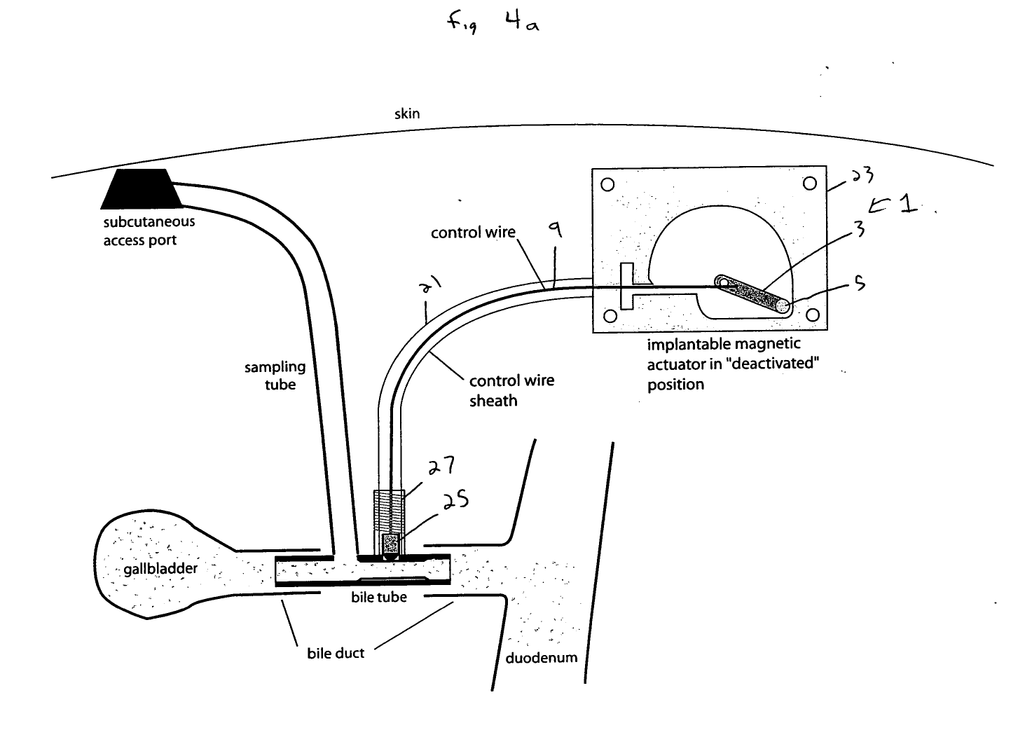

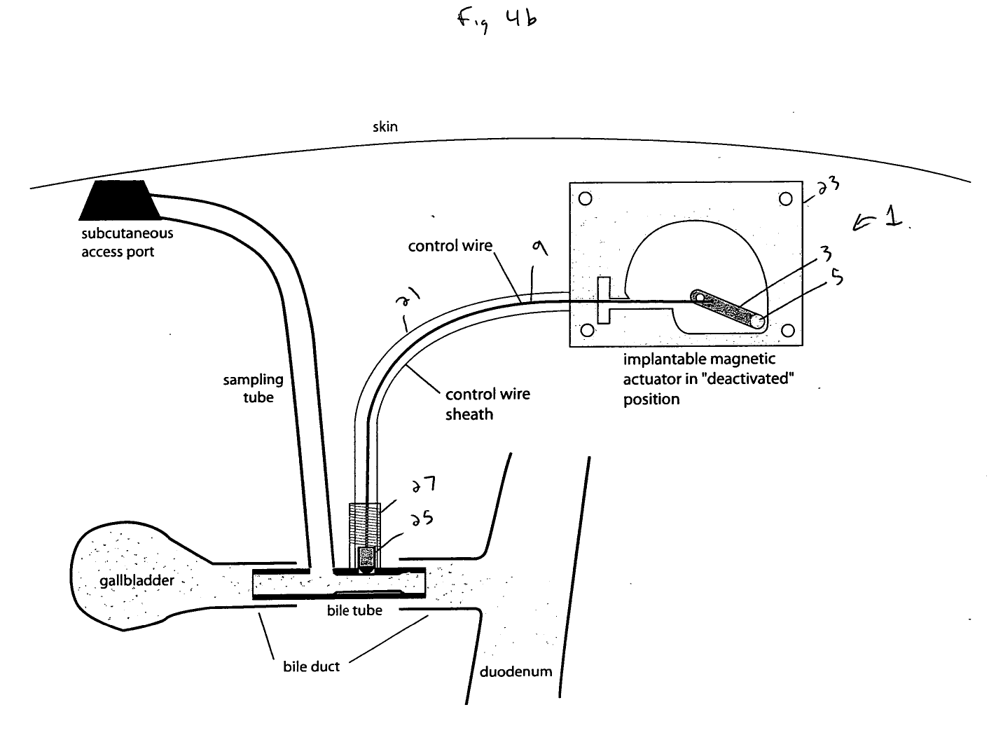

FIG. 4a is a schematic of the implantable, magnetic actuator 1 in use with a medical device intended for bile collection in a research animal. The mechanical switch 3 is shown in a “deactivated” position wherein the connector 9 is withdrawn toward the mechanical switch 3 resulting in the activation means being in the “deactivated” position, located proximally to the mechanical switch 3. The activation means 25 are shown as a plug connected at end of the connector 9.

In this embodiment, the sheath 21 has a spring 27 that is compressed when the mechanical switch 3 is in a “deactivated” position. In the “deactivated” position, the activation means 25, the plug, is withdrawn towards the mechanical switch 3, compressing the spring, and bile is allowed to flow in the normal direction from the gallbladder to the duodenum through the artificial bile tube that has been placed in the bile duct. FIG. 4b illustrates the implantable, magnetic actuator 1 in an “activated” position, wherein the mechanical switch 3 has changed positions and the connector 9 extends towards the activation means 25 thereby forcing the activation means 25, the plug, to move towards the artificial bile tube and allowing the spring to relax. As the activation means 25 move, the artificial bile tube is occluded thereby diverting the flow of bile away from the duodenum towards a subcutaneous access port where the bile is sampled.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed:

1. An implantable, magnetic actuator comprising:
a mechanical switch;
a magnet attached to the mechanical switch;
an external user component which is used externally of the animal's or human's body to couple to the magnet to cause the mechanical switch to change positions; and
a connector that is connected to the mechanical switch at one end and at the opposite end to a mechanism, component, device or surrounding bodily tissue or body part which when the mechanical switch changes positions causes the connector to effect a force on such mechanism, component, device or surrounding bodily tissue.

2. An implantable, magnetic actuator of claim 1, wherein the mechanical switch is enclosed within housing.

3. An implantable, magnetic actuator of claim 2, wherein the connector has a sheath that is concentric to the connector and provides a protective barrier and passageway for the connector to pass.

4. An implantable, magnetic actuator of claim 1, wherein the mechanical switch has a locking mechanism to lock the position of the mechanical switch.

5. An implantable, magnetic actuator of claim 4, wherein the locking mechanism includes a spring connected to the connector.

6. An implantable, magnetic actuator of claim 1, wherein the mechanical switch is inverted where the “deactivated” and “activated” positions are reversed.

7. An implantable, magnetic actuator comprising:
a mechanical switch;
a magnet attached to the mechanical switch;
an external user component which is used externally of the animal's or human's body to couple to the magnet to cause the mechanical switch of the implantable, magnetic actuator to change positions;
a connector that is connected at one end to the mechanical switch; and
activation means, which connect to the connector, for actuating surrounding bodily tissue, intervening in or monitoring the body's activities, or actuating a device that actuates surrounding bodily tissue or intervenes in or monitors the body's activities.

8. An implantable, magnetic actuator of claim 7, wherein the mechanical switch is enclosed within housing.

9. An implantable, magnetic actuator of claim 8, wherein the connector has a sheath that is concentric to the connector and provides a protective barrier and passageway for the connector to pass.

10. An implantable, magnetic actuator of claim 7, wherein the mechanical switch has a locking mechanism to lock the position of the mechanical switch.

11. An implantable, magnetic actuator of claim 10, wherein the locking mechanism inclues a spring connected to the connector.

12. An implantable, magnetic actuator of claim 7, wherein the mechanical switch is inverted where the “deactivated” and “activated” positions are reversed.
